SEQUENCE LISTING (12 sequences)

SEQ ID NO: 1 - RNA, 41 nt, artificial
  oligonucleotide
aaagcuaagcggaccgagucgacugcaucgucaugaaaaaa

SEQ ID NO: 2 - DNA, 41 bp, artificial
  oligonucleotide
ttttttcatgacgatgcagtcgactcggtccgcttagcttt

SEQ ID NO: 3 - DNA, 41 bp, artificial; identical to SEQ ID NO: 2
  oligonucleotide

SEQ ID NO: 4 - DNA, 41 bp, artificial
  oligonucleotide
aaagctaagcggaccgagtcgactgcatcgtcatgaaaaaa

SEQ ID NO: 5 - RNA, 41 nt, artificial
  oligonucleotide
uuuuuucaugacgaugcagucgacucgguccgcuuagcuuu

SEQ ID NO: 6 - DNA, 41 bp, artificial; identical to SEQ ID NO: 2
  oligonucleotide

SEQ ID NO: 7 - RNA, 41 nt, artificial; identical to SEQ ID NO: 1
  oligonucleotide

SEQ ID NO: 8 - DNA, 23 bp, artificial
  oligonucleotide
taaaggcgcgcccgcatgcttta

SEQ ID NO: 9 - RNA, 23 nt, artificial
  oligonucleotide
uaaaggcgcgcccgcaugcuuua

SEQ ID NO: 10 - RNA, 23 nt, artificial
  oligonucleotide
uaaagcaugcgggcgcgccuuua

SEQ ID NO: 11 - DNA, 23 bp, artificial
  oligonucleotide
taaagcatgcgggcgcgccttta

SEQ ID NO: 12 - DNA, 23 bp, artificial
  oligonucleotide
taaagcatgcgggcgcgccttta

(19) United States
(12) Patent Application Publication
(10) Pub. No.: US 2005/0053990 A1
(43) Pub. Date: Mar. 10, 2005

(54) Cleavage of RNA by restriction endonucleases

(76) Inventors: Richard J. Roberts, Wenham, MA (US); Shawn Stickel, Reading, MA (US)

Correspondence Address: NEW ENGLAND BIOLABS, INC., 32 TOZER ROAD, BEVERLY, MA, 01915, US

(21) Appl. No.: 10/930,204

(22) Filed: Aug. 31, 2004

Related U.S. Application Data
(60) Provisional application No. 60/501,182, filed on Sep. 8, 2003.

(51) Int. Cl. C12Q 1/68; C12P 19/34

(52) U.S. Cl. 435/6; 435/91.2

(57) ABSTRACT

Methods of and uses for cleaving RNA/DNA duplexes with restriction endonucleases are provided as well as methods for determining whether a restriction endonuclease is capable of such cleavage.

### CROSS REFERENCE

This application claims priority from U.S. application Ser. No. 60/501,182 filed Sep. 8, 2003, herein incorporated by reference.

### BACKGROUND OF THE INVENTION

There are several reports in the literature of interactions between restriction enzymes and RNA/DNA duplexes. However, none of these have critically examined the precise nature of cleavage other than to report that the DNA strand of an RNA/DNA duplex is sometimes cleaved at least partially. (Molloy and Symons, Nucleic Acids Res. 8:2939-2946 (1980); Nazarenko, et al., Bioorg. Khim. 13:928-933 (1987); Babkina, et al., Mol. Biol. (Mosk) 34:1065-1073 (2000); Kim, et al., Gene 203:43-49 (1997); Krynetskaya, et al., Biochemistry (Mosc) 63:1068-73 (1998)).

Nazarenko et al. (1987) reported that BamHI cleaved RNA in a small percentage of nucleic acid molecules where the nucleic acid molecules consisted of one strand in which DNA was joined to an RNA having a BamHI cleavage site located in the region of the junction and a second strand consisting entirely of DNA. However, the low level of cleavage activity observed for the small fragment of RNA examined undermined the utility of restriction endonucleases for analyzing RNA structure in large or small RNA molecules. No activity was found for Sau3AI.

Molloy et al (1980) reported that cleavage of the RNA strand occurred, but they did not discriminate between specific cleavage by the restriction enzyme and general cleavage by contaminants. This omission was significant because at the time, it was well known that preparations of restriction endonucleases contained non-specific ribonucleases. Therefore, without evidence to the contrary, it would have been assumed that cleavage of RNA resulted from non-specific ribonuclease activity. Consequently, when an RNA fragment is required, other methods have been used. For example, partial RNase H digestion of large RNA produces fragments of varied sizes and non-precise ends. Alternatively, chemical synthesis can be used to create short oligonucleotides. This approach however is costly, time consuming and is limited to oligonucleotides of less than 50 nucleotides in length.

There is considerable interest by nucleic acid biochemists in the possibility of cleaving RNA molecules in a site-specific fashion much as restriction enzymes are used to cleave DNA. RNA molecules play diverse roles in cells although how and why they perform particular functions remains uncertain in many cases. The ability to achieve site-specific cleavage of RNA would permit a degree of manipulation of RNA hitherto not possible.

### SUMMARY OF THE INVENTION

In an embodiment of the invention, a method is provided for cleaving an RNA/DNA duplex where the method includes the steps of: combining a restriction endonuclease, isochizomer or modification thereof, with an RNA/DNA duplex in a mixture, wherein the restriction endonuclease, isoschizomer or modification thereof is capable of cleaving the RNA/DNA duplex to form a plurality of RNA/DNA duplex fragments of specific sizes with defined ends; and cleaving the RNA/DNA duplex. Examples of restriction endonucleases that may be used in this method include AvaII, Cac8I, BstI, SfaNI and Sau3AI.

Restriction endonuclease may be modified to selectively cleave only RNA in the RNA/DNA duplexes. Sau3AI is an example of a restriction endonuclease that does not require modification to cleave only RNA. Alternatively, metal ions other than magnesium can be included in the mixture for inhibiting DNA duplex cleavage by the restriction endonuclease.

The minimum size of the duplex prior to cleavage is defined by the size of the recognition site plus about two additional nucleotides. There is no limit on the maximum length. The product of cleavage can then be denatured to generate single stranded RNA fragments of defined size and ends.

In an embodiment of the invention, a method is provided for determining whether a restriction endonuclease is in fact capable of cleaving an RNA only within an RNA/DNA duplex. The method includes the steps of: obtaining a labeled RNA/DNA oligonucleotide duplex; cleaving the duplex with a restriction endonuclease; and analyzing the products of the reaction by size separation to determine whether the restriction endonuclease is capable of cleaving the RNA in the duplex in the absence of ribonuclease activity.

In an embodiment of the invention, a method is provided for detecting a pathogenic RNA virus. The method includes the steps of: hybridizing viral RNA in a biological sample with a single-stranded DNA (ssDNA) fragment; cleaving the RNA/DNA duplex with one or more restriction endonucleases, isoschizomers or modifications thereof having known recognition and cleavage specificities for the RNA/DNA duplex to produce RNA fragments having characteristic fragment sizes in an RNA profile; and detecting the pathogenic RNA virus from the RNA profile. For example, the restriction endonuclease can be at least one of AvaIl, Cac8I, BtsI, SfaNI and Sau3AI.

In an embodiment of the invention, a method of treating a subject infected with an RNA containing virus is provided. The method includes administering to a subject, an effective dose of one or more restriction endonucleases isoschizomers or modifications in a pharmaceutical formulation. The formulation includes one or more restriction endonucleases, isoschizomers or modifications thereof which are capable of cleaving RNA/DNA duplexes and reducing the virus load in the affected subject. The restriction endonuclease used in the above method may for example be at least one of AvaII, Cac8I, BtsI, SfaNI and Sau3AI. An example of an RNA-containing virus is Human Immunodeficiency Virus.

In an embodiment of the invention, a method is provided for obtaining a double-stranded RNA (dsRNA) fragment having a defined length and terminal sequence. The method includes the steps of: cleaving an RNA/DNA duplex with a restriction endonuclease, an isoschizomer or modification thereof having known cleavage specificity; denaturing the cleaved RNA/DNA duplex so that the RNA hybridizes to itself or a second RNA to form an RNA/RNA duplex; and obtaining the double stranded RNA fragment having a defined length and terminal sequence. Examples of restriction endonucleases for use in the above method include the restriction endonucleases Ava II, Cac8I, SfaNI, BtsI and Sau3AI.

In an embodiment of the invention, a method of gene silencing is provided that includes the steps of: cleaving an RNA/DNA duplex with one or more restriction endonucleases, isoschizomers or modifications thereof; denaturing the duplex to provide a single-stranded RNA (ssRNA); permitting the ssRNA to reanneal into a hairpin or RNA duplex; transfecting target cells with the RNA duplex; and obtaining gene silencing. Examples of restriction endonucleases for use in the above method include the restriction endonuclease are Ava II, Cac8I, SfaNI, BtsI and Sau3AI.

In an embodiment of the invention, a method of mapping long RNA molecules is provided that includes the steps of: hybridizing a long RNA molecule to DNA oligonucleotides containing one or more endonuclease cleavage sites to form an RNA/DNA duplex; cleaving the RNA/DNA duplex with one or more restriction endonucleases to generate fragments; denaturing the duplex and separating the cleaved RNA fragments by size to form an RNA profile; and mapping the long RNA molecules from the RNA profile.

In an embodiment of the invention, a method is provided for detecting alternative spliced forms of messenger RNAs (mRNAs), that include the steps of: hybridizing an mRNA molecule to DNA oligonucleotides containing one or more endonuclease cleavage sites to form an RNA/DNA duplex; cleaving the RNA/DNA duplex with one or more restriction endonucleases; denaturing the duplex and separating the cleaved RNA fragments by size to form an RNA profile; and detecting the alternative spliced forms of the mRNAs from the RNA profile.

In an embodiment of the invention, a method is provided for generating RNA primers for DNA polymerase or reverse transcriptase, that include the steps of: hybridizing an RNA molecule to DNA oligonucleotides containing one or more endonuclease cleavage sites to form an RNA/DNA duplex; cleaving the RNA/DNA duplex with one or more restriction endonucleases to generate fragments; denaturing the RNA/DNA duplex and separating the cleaved RNA fragments by size; and generating RNA primers for DNA polymerase or reverse transcriptase.

In an embodiment of the invention, a method is provided for RNA sequence shuffling for expressing a novel protein. The method includes the steps of: hybridizing an RNA molecule to DNA oligonucleotides containing one or more endonuclease cleavage sites; cleaving RNA/DNA duplex with one or more restriction endonucleases to generate RNA/DNA fragments; denaturing the duplex and separating the cleaved RNA fragment by size; and ligating a sized RNA fragment to a second sized RNA fragment in the presence of RNA ligase to form shuffled RNA sequences for expressing a novel protein.

### BRIEF DESCRIPTION OF THE DRAWINGS

FIGS. 1-3 show the results of cleavage of duplexes formed from the following single-stranded oligonucleotides. The cleavage site of AvaII is 5′ . . . GG A/T CC . . . 3′ with cleavage between G and G. The cleavage site of Cac8I is 5′ . . . GCNNGC . . . 3′ with cleavage between the two Ns.

| | |
|---|---|
| A. RNA: | |
| 5′ AAAGCUAAGCGGACCGAGUCGACUGCAUCGUCA | (SEQ ID NO: 1) |
| UGAAAAAA-Fam-3′ | |
| B. DNA: | |
| 5′-TTTTTTCATGACGATGCAGTCGACTCGGTCCGC | (SEQ ID NO: 2) |
| TTAGCTTT-Fam-3′ | |
| C. DNA: | |
| 5′-TTTTTTCATGACGATGCAGTCGACTCGGTCCGC | (SEQ ID NO: 3) |
| TTAGCTTT-3′ (no label) | |
| D. DNA: | |
| 5′-AAAGCTAAGCGGACCGAGTCGACTGCATCGTCA | (SEQ ID NO: 4) |
| TGAAAAAA-Fam-3′ | |
| E. RNA: | |
| 5′-UUUUUUCAUGACGAUGCAGUCGACUCGGUCCGC | (SEQ ID NO: 5) |
| UUAGCUUU-Fam-3′ | |
| F. DNA: | |
| 5′-TTTTTTCATGA(m5C)GATGCAGTCG(n6A)CT | (SEQ ID NO: 6) |
| CGGTC(m5C)GCTTAGCTTT-Fam-3′ | |
| G. RNA: | |
| 5′-Fam-AAAGCUAAGCGGACCGAGUCGACUGCAUC | (SEQ ID NO: 7) |
| GUCAUGAAAAAA-3′ | |

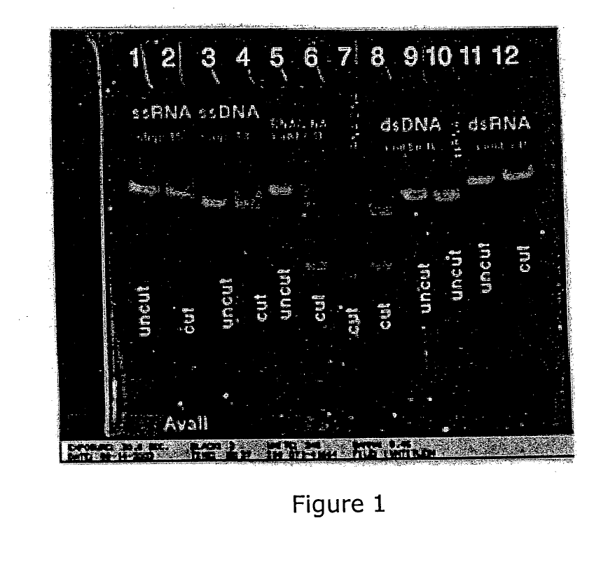

FIG. 1 shows a TBE polyacrylamide gel in which DNA duplexes, RNA/RNA duplexes and RNA/DNA duplexes are cleaved with AvaII, where lanes 1-12 contain the following:

| Lane | DNA | RNA | Enzyme |
|---|---|---|---|
| 1. | | A | none |
| 2. | | A | AvaII |
| 3. | B | | none |
| 4. | B | | AvaII |
| 5. | A | B | none |
| 6. | A | B | AvaII |
| 7. | A | C | AvaII |
| 8. | B, D | | AvaII |
| 9. | B, D | | none |
| 10. | | E | none |
| 11. | | A, E | none |
| 12. | | A, E | AvaII |

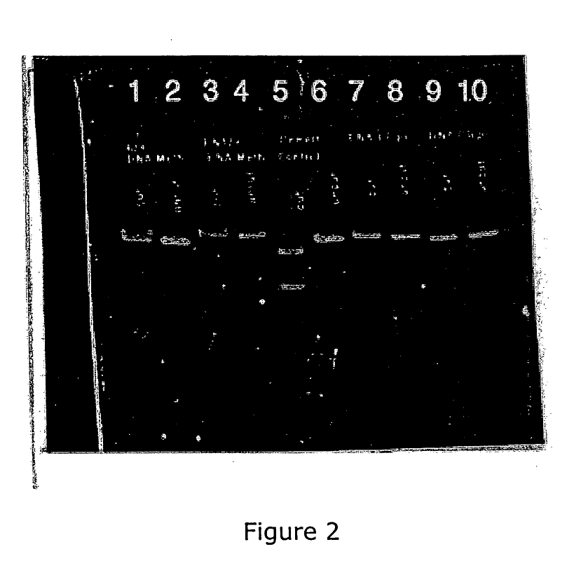

FIG. 2 shows a TBE polyacrylamide gel in which methylated DNA/DNA duplexes (m5C and n6A), unmethylated DNA duplexes, methylated RNA/DNA duplexes and unmethylated RNA/DNA duplexes are cleaved with AvaII as follows:

| Lane | DNA | RNA | Enzyme |
|---|---|---|---|
| 1. | D, F | | AvaII |
| 2. | D, F | | none |
| 3. | F | G | AvaII |
| 4. | F | G | none |
| 5. | B, D | | none |
| 6. | B, D | | AvaII |
| 7. | | G | AvaII |
| 8. | | G | none |
| 9. | B | | AvaII |
| 10. | B | | none |

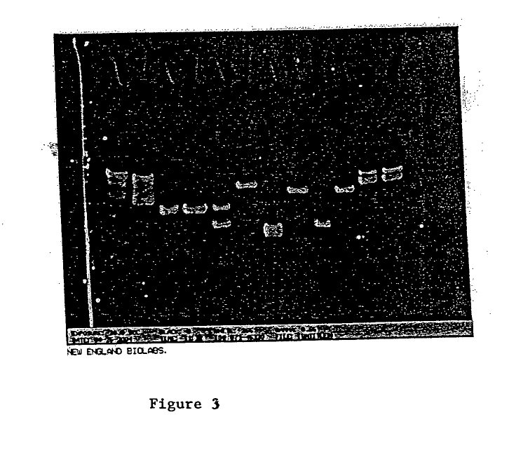

FIG. 3 shows a TBE polyacrylamide gel in which DNA duplexes, RNA duplexes and RNA/DNA duplexes are cleaved with Cac8I as follows:

| | |
|---|---|
| Lane DNA RNA Enzyme | |
| H. DNA: 5′-FAM-TAAAGGCGCGCCCGCATGCTTTA-3′ | (SEQ ID NO: 8) |
| I. RNA: 5′-FAM-UAAAGGCGCGCCCGCAUGCUUUA-3′ | (SEQ ID NO: 9) |
| J. RNA: 5′-FAM-UAAAGCAUGCGGGCGCGCCUUUA-3′ | (SEQ ID NO: 10) |
| K. DNA: 5′-TAAAGCATGCGGGCGCGCCTTTA-3′ | (SEQ ID NO: 11) |
| L. DNA: 5′-FAM-TAAAGCATGCGGGCGCGCCTTTA-3′ | (SEQ ID NO: 12) |

| Lane | DNA | RNA | Enzyme |
|---|---|---|---|
| 1. | | J | Cac8I |
| 2. | | J | none |
| 3. | H | | Cac8I |
| 4. | H | | none |
| 5. | L | I | Cac8I |
| 6. | L | I | none |
| 7. | H, L | | Cac8I |
| 8. | H, L | | none |
| 9. | K | I | Cac8I |
| 10. | K | I | none |
| 11. | | I, J | Cac8I |
| 12. | | I, J | none |

### DESCRIPTION OF THE EMBODIMENTS

We have shown that more than one restriction endonuclease is capable of specific RNA cleavage and have established that RNA cleavage is the result of the restriction endonuclease cleavage and not contaminant enzymes or other factors. The extent of cleavage is significant, for example, at least 10% and as much as 100% of RNA substrate may be cleaved under the exemplified conditions.

A plurality of restriction endonucleases have here been shown to be capable of cleaving RNA in RNA/DNA duplexes although this property is not inherent in the universe of restriction endonucleases. It is taught here that restriction endonuclease cleavage of RNA/DNA duplexes have precise ends corresponding to the cleavage site of the restriction endonuclease. RNA is expected to be size limited only at the lower end of the range, typically 2-8 nucleotides longer than the length of the recognition sequence of the restriction enzyme. For example, for the 5-nucleotide long AvaII recognition sequence, an oligonucleotide containing 1-4 additional nucleotides on either side of the recognition sequence will likely be required for cleavage to be effective. While not wishing to be limited by theory, a minimum sequence length may be that number of nucleotides which enable the restriction endonuclease to bind to the duplex in order to cleave the RNA.

The methods described herein can be used to evaluate any restriction endonuclease for RNA cleavage activity. Examples of restriction endonucleases can be found in the New England Biolabs catalog (Beverly, Mass.) or in REBASE® (and see also Roberts et al. Nucleic Acids Res. 31:418-420 (2003)). The substrates used in the examples are short duplexes prepared by annealing a synthetic ribo-oligonucleotide (RNA strand) with a synthetic deoxyribo-oligonucleotide (DNA strand) (Example 1). Both strands carry fluorescent tags, at either their 3′ end or 5′ end to facilitate detection. The synthetic oligonucleotides are designed to contain many restriction enzyme sites for convenience where one duplex can be used as a substrate for many enzymes. The experimental conditions for these digestions utilize the buffers and conditions described by the manufacturer (New England Biolabs, Beverly, Mass.). Following digestion, the reaction products are assayed using both denaturing and non-denaturing gels. Controls for these experiments include:
a) testing for cleavage of ssRNA to determine the absence of non-specific ribonucleases which could otherwise confuse the result.
b) testing for cleavage of the ssDNA since this is known to occur in some cases and might confuse interpretation if annealing were incomplete.
c) digesting a dsDNA oligonucleotide of the same sequence to provide markers for the cleavage products.
d) testing for cleavage of a duplex in which the RNA strand is fluorescently tagged, but the DNA strand is unlabelled to render unambiguous the specific cleavage effects on the RNA strand.

Reaction products were separated on a 20% TBE gel. Cleavage was detected using fluorophore labeling of a synthetic RNA at its 3′ or 5′ end and optionally fluorophore labeling of the DNA strand at its 3′ end. Table 1 shows examples of endonucleases that were identified as being able to cleave RNA in an RNA/DNA duplex in the established absence of RNAase.

TABLE 1

Results of restriction
endonuclease cleavage of RNA/DNA duplexes

| | Recognition | % RNA strand | % DNA strand |
|---|---|---|---|
| Enzyme | Sequence | cleaved | cleaved |
| AvaII | GGWCC | 100 | 100 |
| BtsI | GCAGTG | 70 | 100 |
| Cac8I | GCNNGC | 100 | 100 |
| SfaNI | GCATC | 100 | 100 |
| Sau3AI | GATC | 80 | 0 |

RNA fragments created by cleavage with enzymes such as in Table 1 will typically termini corresponding to the nucleotides adjacent to the cleavage site on either side and on either strand. Details of the precise cleavage site for any particular restriction endonuclease are readily ascertained by using RebaseR (New England Biolabs, Inc., Beverly, Mass.).

Selective cleavage of RNA and not DNA in an RNA/DNA duplex by restriction endonucleases may be achieved naturally (as for Sau3AI) or possibly under predetermined conditions such as: in the presence of inhibitors such as metal ions other than magnesium; changes in pH; changes in temperature; or mutations in the endonuclease or methylation of non-cognate bases (i.e., those bases not usually used for protection).

(a) The effects of metal ions, pH and temperature on restriction enzyme cleavage of the DNA strand in an RNA/DNA duplex can be tested under otherwise standard conditions according to the assays described in Example 1 to determine differential effects on RNA versus DNA strand cleavage. Buffers that include any of metal ions such as Mn²⁺, Ca²⁺, Co²⁺, Ni²⁺ etc., pH or temperature can be tested. Individual metal ions may be substituted for Mg²⁺ ions at 1-20 mM concentrations. Alternatively the pH of the standard buffers may be varied from pH 5.0 to pH 10.0 using different salts appropriate to each pH. Tris-acetate buffers can be used for the lower pHs and Tris-HCl buffers for the higher pHs.

(b) The effect of methylation of the DNA strand has been tested to further confirm that cleavage of RNA is specific. Example 3 shows how AvaII cleavage of the RNA strand is blocked by methylation of the DNA strand on the 5° C. of the DNA strand at CCTGG in the RNA/DNA duplex (FIG. 2). We expect this to be a general phenomenon.

(c) The exact site of cleavage within the RNA strand can be determined by displaying the cleaved product on a denaturing gel, using partial digests of the starting RNA strand as markers, for example, as prepared with NaOH, ribonuclease T1 and pancreatic ribonuclease. Initial studies performed as described suggest that cleavage occurs at a canonical site

(d) Inhibition of cleavage of DNA in an RNA/DNA duplex may be achieved by means of mutant enzymes that are defective in DNA cleavage. The rationale is that RNA cleavage is chemically much easier than DNA cleavage and so the latter may be inhibited while still permitting the former. Mutations would initially target the known active site for some of the enzymes for which crystal structures are available. The experimental protocols to be used for mutagenesis would utilize standard protocols such as described in the Molecular Cloning Manual (ed. Sambrook et al., Cold Spring Harbor, N.Y. (2003)).

The length of a synthetic DNA strand required to hybridize to a polyribonucleotide so as to render it susceptible to cleavage can be determined. The minimum length is predicted to be that number of nucleotides sufficient for the restriction endonuclease to recognize a specific sequence and to bind to the nucleic acid. For double-stranded DNA (dsDNA), there is usually a requirement for at least 1-4 flanking nucleotides on either side of the recognition sequence for efficient cleavage. The maximum size may be essentially limited only by synthesis methodology. Reaction conditions described in Example 1 are used and the size of the substrate varied as desired.

The Examples utilize synthetic oligonucleotides as substrates. However, approximately 30 years of research into restriction endonucleases teach that restriction endonucleases do not differentiate between short DNA and long DNA as cleavage substrates. Consequently, it is appropriate to extrapolate from the findings using RNA/DNA oligonucleotides as substrates to RNA/DNA duplexes of any length greater than the minimum described above.

Use of Site-Specific RNA Cleavage

Restriction endonucleases that are capable of cleaving RNA/DNA duplexes may be used for any application involving RNA that has been previously identified for DNA. Most importantly, restriction endonucleases provide physical landmarks for characterizing a piece of RNA (or DNA) where the cleavage products provide a fragment profile on a separation medium such as a gel that is a fingerprint for the uncleaved substrate.

DNA fragments that are the product of restriction endonuclease cleavage or are synthesized chemically having defined termini can be used to hybridize with long or short RNA for precise cleavage of the RNA. An advantage of forming a duplex over a short region of a nucleic acid is that the rate of annealing is rapid and targeted cleavage can be achieved efficiently. The resulting cleaved product may be used for characterizing the RNA.

RNA fragments resulting from cleavage of RNA/DNA duplexes by endonucleases have multiple uses.

(a) Long RNAs may be mapped in a manner that is similar to DNA mapping.

(b) Alternatively spliced forms of mRNAs can be detected by examining cleavage profiles.

(c) RNA primers for DNA polymerase or reverse transcriptase reactions can be formed using for example modified restriction endonucleases. Modified restriction endonucleases that do not cleave DNA could be particular useful to generate ssRNA molecules as they would allow the short DNA oligonucleotides to be recycled during the cleavage reaction.

(d) Long RNAs can be manipulated in a way that is presently impossible.

(e) The effects of gene shuffling can be achieved by permutation in fragments that constitute an mRNA transcript. For instance, RNA fragments after endonuclease cleavage of an RNA/DNA duplex and followed by denaturation may be joined together using an RNA ligase so as to generate novel shuffled combinations. These shuffled combinations may be used in in vitro evolution experiments. Such RNA fragments may also be used to analyze the function or specific sequence content of portions of RNA molecules in ways that are currently not possible.

(f) Specific RNA fragments can be generated by cleavage of RNA/DNA duplexes for use in gene silencing (RNAi). For example, long synthetic RNA molecules are cleaved into precise pieces of desired length and sequence. To this end, RNA/DNA duplexes may be selectively cleaved with restriction enzymes to generate suitable specific fragments for RNAi.

(g) RNA viruses strains can be identified for epidemiological studies by generating characteristic RNA profiles of the viruses strains using restriction endonucleases and for therapeutic or preventative treatment. Examples of the use of restriction endonucleases as diagnostic reagents include the diagnosis of various strains of SARS. SARS is caused by a mutant coronavirus, which is a cytoplasmic ssRNA virus. Characterization of coronavirus isolates from various sources by RNA profiling could prove very useful in epidemiological studies.

There are many RNA viruses that are pathogens and occur in a wide range of different infectious strains. Perhaps the most well known and highly variable RNA virus is the rhinovirus that causes the common cold. Other RNA viruses include Picornaviruses (poliovirus) Arenaviridae, Bunyaviridae, Flaviviridae, (Yellow fever, Hepatitis C&G), Orthomyxoviridae (Influenza) Paramyxoviridae (Mumps), Reoviridae, Retroviridae (AIDS) Rhabdoviridae (Rabies), Vesicular stomatitis virus, Togaviridae, Filoviridae (Ebola fever).

(h) Restriction endonucleases can be used as therapeutic agents. It is proposed here that the ability to cleave an RNA/DNA duplex might be useful to inhibit viruses during infection. For example, when retroviruses infect cells, they release their RNA genome to create a DNA copy thereby making an RNA/DNA duplex. An effective dose of a restriction endonuclease may be used to cleave these viral duplexes such that both the DNA and the RNA strand of the infectious RNA/DNA duplex molecule made by reverse transcription are cleaved and then destroyed in order to slow down infection. For example, AvaII could be used for site-specific cleavage of RNA/DNA duplex products produced during HIV infection. In these circumstances, it is desirable to inhibit or inactivate the endonuclease capability for cleaving dsDNA by mutating the endonuclease or alternatively by confining the endonuclease to the cytoplasm of targeted cells or extracellular space.

The endonuclease may be formulated in a manner that is suited for its delivery. The formulation should be pharmacologically acceptable and may include an excipient according to the art. The mode of delivery of restriction endonuclease to infected cells, tissues or body fluids of a subject (where the subject is an animal including a human) include the following methods known in the art such as oral, intravenous, intramuscular, transdermal or transmucosal routes of delivery. The restriction endonuclease may be delivered to target cells, packaged in a micellar structure such as a liposome, encapsulated in a viral capsid such as Adeno-associated virus, or targeted to cells by conjugating to carrier molecules. Alternatively, the gene encoding the restriction endonuclease can be integrated into a viral vector so that the gene can be delivered by infection or delivered by other gene therapy delivery mechanisms. Examples of viral vectors include retroviruses, herpes viruses, adenoviruses and vaccinia viruses.

An effective dose of the restriction endonuclease would be the amount sufficient to slow or reverse development of disease by reduction of viral load.

All references cited above and below are herein incorporated by reference.

The present invention is further illustrated by the following Examples. These Examples are provided to aid in the understanding of the invention and are not construed to be a limitation thereof.

### EXAMPLE 1

### Protocol for Testing for Cleavage of RNA/DNA Duplexes with Restriction Enzymes

Oligonucleotides were synthesized using an Applied Biosystems (Foster City, Calif.) 394 RNA/DNA Synthesizer and labelled with Fam. All the FAM labeled RNA was labeled specifically with the isomer 6-FAM. (Fam is an amine reactive fluorescein ester of carboxyfluorescein). Fluorescein phosphoramidites and RNA/DNA amidites were purchased from Glen Research, Sterling, Va.

The fluorescent tag was attached to the 3′ end (A, B, D, E and F) and to the 5′ end (G) of synthetic oligonucleotides (A)-(G) for visualization on polyacrylamide gels. The 7 synthetic oligonucleotides used herein are listed in the Description of the Figures (SEQ ID NOS:1-7).

The oligonucleotides were resuspended in TE (10 mM Tris, 1 mM EDTA pH 7.0) buffer or water and their concentration adjusted to 10 μM concentration.

RNA and DNA oligos were annealed together to form an RNA/DNA duplex(A/B and A/C). DNA oligos were annealed together to form the control dsDNA (B/D and D/F (methylated)) and RNA oligos were annealed together to form an RNA duplex (A/E).

The annealing reaction for the RNA/DNA duplexes, DNA duplexes and RNA duplexes was as follows:
5 μl oligo top strand (10 μM)
5 μl oligo bottom strand (10 μM)
5 μl NEB (New England Biolabs, Inc., Beverly, Mass.) Buffer 4 (20 mM Tris-Acetate, 10 mM magnesium acetate, 50 mM potassium acetate, 1 mM DTT (pH 7.9))
35 μl water

The mixture was heated to 90° C. for 5 minutes and slowly cooled to room temperature.

Using the annealed oligonucleotide duplexes, restriction endonucleases were tested to determine if cleavage of RNA occurred. The absence of RNase was confirmed by incubating ssRNA oligonucleotides with the test endonuclease and determining whether any cleavage occurred using polyacrylamide gel electrophoresis.

The following protocol utilizes AvaII. However AvaII can be substituted for any restriction endonuclease in the amount and under the conditions recommended for DNA duplex cleavage.

Digestion of RNA/DNA, dsDNA, or dsRNA with AvaII restriction enzyme:
1 μl annealed oligo combinations
2 μl NEBuffer 4 (recommended for AvaII)
2 μl AvaII (@10 μ/μl)
15 μl water

The mixture was digested for 4 hours at 37° C.

4 μl 30% glycerol was added to the samples.

Digested and undigested annealed oligo combinations were run on 20% TBE polyacrylamide gels (Invitrogen Catalog #EC6315, (Carlsbad, Calif.) for 1.5 hours at 100 volts.

The results obtained for AvaII digestion are provided in FIG. 1.

The results show that the RNA in the RNA/DNA duplex (A/B or A/C) was cleaved by AvaII as seen by the comparison of digested and undigested duplex (FIG. 2, Lanes 5-7), DNA/DNA was cleaved by AvaII as seen by the comparison of digested and digested dsDNA (FIG. 2, B/D, Lanes 8 and 9) but RNA/RNA (A/E) was not cleaved by AvaII as seen by the comparison of digested and digested dsRNA (FIG. 2, Lanes 11 and 12)

### EXAMPLE 2

### Inhibition of Cleavage of RNA/DNA Duplex by Methylation

RNA top strand was annealed to methylated DNA bottom strand (F/G) as described in Example 1. In addition, DNA top strand was annealed under the conditions described in Example 1 to methylated DNA bottom strand (D/F) for use as a control.

The recognition sequence of AvaII is GGWCC. The annealed oligo combination has a methylated final C of the DNA strand.

| | |
|---|---|
| (D) | |
| AAAGCUAAGCGGACCGAGUCGACUGCAUCGUCAUGA | (SEQ ID NO: 4) |
| AAAAA | |
| (B) | |
| TTTCGATTCGCCTGGCTCAGCTGACGTAGCAGTACT | (SEQ ID NO: 2) |
| m m m | |
| TTTTT | |

AvaII digestion is known to be blocked with the methylation of the final C in the cleavage site within dsDNA (see REBASE®). AvaII was digested according to Example 1 and run on TBE gels (FIG. 2). Digested and undigested oligonucleotide combinations were run side by side to compare results. It was found that indeed the methylation of the final C blocked cleavage of DNA by AvaII. Both DNA/DNA (FIG. 2, Lanes 1 and 2) and RNA/DNA duplex (FIG. 2, Lanes 3 and 4) were blocked by the methylation of the final C in the DNA strand while unmethylated dsDNA was digested (FIG. 2, Lanes 5 and 6).

### EXAMPLE 3

### The RNA Strand in an RNA/DNA Duplex is Digested with Restriction Endonuclease

An RNA/DNA duplex was made with a fluorescent tag on the RNA and unlabeled DNA strand as described in Example 1 and digested with AvaII. Digested and undigested duplex were run side by side on 20% polyacrylamide TBE gel (as described in Example 1).

The results are shown in FIG. 1 where the RNA strand of the RNA/DNA duplex was digested with AvaII. (FIG. 1, Lane 7).

### EXAMPLE 4

### Verification that RNA Cleavage was not a Product of RNAse Digestion nor of Cleavage of ssDNA

Single-stranded RNA and ssDNA oligos were digested with AvaII as described in Example 1 and the digested and undigested oligonucleotides were run side by side on 20% polyacrylamide TBE gel (as described in Example 1). The results are shown in the summary of FIG. 2.

The results showed that there are no RNAses present in the AvaII digestion. (FIG. 1, Lanes 1 and 2) and AvaII did not digest ssDNA. (FIG. 1, Lanes 3 and 4)

### EXAMPLE 5

### Digestion with Cac8I

Oligonucleotides were synthesized as described in Example 1. All oligonucleotides had the fluorescent tag attached to the 5′ end. The four synthetic oligonucleotides used herein are listed in the Description of the Figures (SEQ ID NOS:8-12)

The oligonucleotides were resuspended in TE (10 mM Tris, 1 mM EDTA pH 7.0) buffer or water and their concentration adjusted to 10 μM concentration.

The following duplexes were tested:

(i) RNA and DNA oligonucleotides were annealed together to form an RNA/DNA duplex (L/I). DNA oligonucleotides were annealed together to form control dsDNA (H/L) and RNA oligonucleotides were annealed together to form an RNA duplex (I/J).

The annealing reactions and subsequent digests were performed as described in Example 1, except that NEBuffer 3 was substituted for NEBuffer 4 in the digestion reactions (New England Biolabs, Inc., Beverly, Mass.).

The results obtained for Cac8I are provided in FIG. 3.

The results show that there are no significant RNAses present in the Cac8I restriction enzyme preparation as judged by comparison of digested and undigested ssRNA (FIG. 3, Lanes 1 and 2). Cac8I does not digest ssDNA as shown by comparison of digested and undigested ssDNA (FIG. 3, Lanes 3 and 4). RNA/DNA duplex was cleaved by Cac8I as shown by comparison of digested and undigested RNA/DNA duplex (FIG. 3, Lanes 5 and 6). The RNA strand of the RNA/DNA duplex was cleaved by Cac8I as shown by comparison of digested and undigested RNA/DNA duplex with only the RNA strand labeled with a fluorescent tag (described in Example 3, as shown in FIG. 3, Lanes 9 and 10). DNA/DNA was cleaved by Cac8I as shown by comparison of digested and undigested dsDNA (FIG. 3, Lanes 7 and 8). RNA/RNA was not cleaved by Cac8I as shown by comparison of digested and undigested dsRNA (FIG. 3, Lanes 11 and 12)

1. A method of cleaving an RNA/DNA duplex, comprising:
(a) combining a restriction endonuclease, isochizomer or modification thereof, with an RNA/DNA duplex in a mixture, wherein the restriction endonuclease, isoschizomer or modification thereof is capable of cleaving the RNA/DNA duplex to form a plurality of RNA/DNA duplex fragments of specific sizes with defined ends; and
(b) cleaving the RNA/DNA duplex.

2. A method according to claim 1, wherein the restriction endonuclease is a modified restriction endonuclease that selectively cleaves RNA in the RNA/DNA duplexes without substantial cleavage of double-stranded DNA (dsDNA).

3. A method according to claim 1, wherein the mixture further comprises metal ions other than magnesium for inhibiting DNA duplex cleavage by the restriction endonuclease.

4. A method according to claim 1, wherein the restriction endonuclease recognizes a specific sequence on the RNA/DNA duplex, such that the size of the RNA/DNA duplex is at least 2 nucleotides longer than the recognition sequence.

5. A method according to claim 1, further comprising: denaturing the duplex to form single-stranded RNA (ssRNA) fragments of defined size and ends.

6. A method according to claim 1, wherein the restriction endonuclease is AvaII.

7. A method according to claim 1, wherein the restriction endonuclease is Cac8I.

8. A method according to claim 1, wherein the restriction endonuclease is BstI.

9. A method according to claim 1, wherein the restriction endonuclease is SfaNI.

10. A method according to claim 1, wherein the restriction endonuclease is Sau3AI.

11. A method for determining whether a restriction endonuclease is capable of cleaving an RNA within an RNA/DNA duplex, comprising:
(a) obtaining a labeled RNA/DNA oligonucleotide duplex;
(b) cleaving the RNA/DNA duplex with a restriction endonuclease; and
(c) analyzing the products of the reaction by size separation to determine whether the restriction endonuclease is capable of cleaving the RNA in the duplex in the absence of ribonuclease activity.

12. A method according to claim 11, wherein the restriction endonuclease has a known DNA cleavage specificity under standard reaction conditions.

13. A method of detecting a pathogenic RNA virus, comprising:
(a) hybridizing viral RNA in a biological sample with a single-stranded DNA (ssDNA) fragment;
(b) cleaving the RNA/DNA duplex with one or more restriction endonucleases, isoschizomers or modifications thereof having known recognition and cleavage specificities;
(c) denaturing the RNA/DNA duplex to produce RNA having characteristic fragment sizes in an RNA profile; and
(d) detecting the pathogenic RNA virus from the RNA profile.

14. A method according to claim 13, wherein the restriction endonuclease is selected from the group consisting of AvaII, Cac8I, BtsI, SfaNI and Sau3AI.

15. A method of treating a subject infected with an RNA-containing virus to reduce viral load, comprising: administering to a subject, an effective dose of one or more restriction endonucleases, isoschizomers or modifications thereof in a pharmaceutical formulation, wherein the one or more restriction endonucleases, isoschizomers or modifications thereof are capable of cleaving RNA/DNA duplexes; and reducing viral load in the subject.

16. A method according to claim 15, wherein the restriction endonuclease is selected from the group consisting of AvaII, Cac8I, BtsI, SfaNI and Sau3AI.

17. A method according to claim 15, wherein the viral pathogen is Human Immunodeficiency Virus.

18. A method of obtaining a double-stranded RNA (dsRNA) fragment having a defined length and terminal sequence, comprising:
(a) cleaving an RNA/DNA duplex with a restriction endonuclease, an isoschizomer or modification thereof having known cleavage specificity;
(b) denaturing the cleaved RNA/DNA duplex so that the RNA hybridizes to itself or a second RNA to form an RNA/RNA duplex; and
(c) obtaining the dsRNA fragment having a defined length and terminal sequence.

19. A method according to claim 18, wherein the restriction endonuclease is Ava II, Cac8I, SfaNI, BtsI and Sau3AI.

20. A method of gene silencing, comprising:
(a) cleaving an RNA/DNA duplex with one or more restriction endonucleases, isoschizomers or modifications thereof;
(b) denaturing the cleaved RNA/DNA duplex to provide a ssRNA;
(c) permitting the ssRNA to reanneal into a hairpin or RNA duplex;
(d) transfecting target cells with the RNA duplex; and
(e) obtaining gene silencing.

21. A method according to claim 20, wherein the restriction endonuclease is selected from: AvaII, Cac8I, BtsI, SfaNI and Sau3AI.

22. A method of mapping a long RNA molecule, comprising:
(a) hybridizing a long RNA molecule to DNA oligonucleotides containing one or more endonuclease cleavage sites to form an RNA/DNA duplex;
(b) cleaving the RNA/DNA duplex with one or more restriction endonucleases;
(c) denaturing the cleaved RNA/DNA duplex and separating the cleaved RNA by size to form an RNA profile; and
(d) mapping the long RNA molecule from the RNA profile.

23. A method of detecting alternative spliced forms of messenger RNAs (mRNAs), comprising:
(a) hybridizing an mRNA molecule to DNA oligonucleotides containing one or more endonuclease cleavage sites to form an RNA/DNA duplex;
(b) cleaving the RNA/DNA duplex with one or more restriction endonucleases;
(c) denaturing the RNA/DNA duplex and separating the cleaved RNA by size to form an RNA profile; and
(d) detecting the alternative spliced forms of the mRNAs from analyzing the RNA profile.

24. A method for generating RNA primers for DNA polymerase or reverse transcriptase, comprising:
(a) hybridizing an RNA molecule to DNA oligonucleotides containing one or more endonuclease cleavage sites to form an RNA/DNA duplex;
(b) cleaving the RNA/DNA duplex with one or more restriction endonucleases;
(c) denaturing the cleaved RNA/DNA duplex and separating the cleaved RNA by size; and
(d) generating RNA primers for DNA polymerase or reverse transcriptase.

25. A method of RNA sequence shuffling for expressing a novel protein, comprising:
(a) hybridizing an RNA molecule to DNA oligonucleotides containing one or more endonuclease cleavage sites;
(b) cleaving RNA/DNA duplex with one or more restriction endonucleases;
(c) denaturing the RNA/DNA duplex and separating the cleaved RNA by size; and
(d) ligating a sized RNA fragment to a second sized RNA fragment in the presence of RNA ligase to form shuffled RNA sequences for expressing a novel protein.
